(19) United States
(12) Patent Application Publication
(10) Pub. No.: US 2016/0135547 A1
(43) Pub. Date: May 19, 2016

(54) Quick On/Off Belt

(71) Applicants: Nathan Kuffrey, Attleboro, MA (US); Daniel Allard, Millbury, MA (US)

(72) Inventors: Nathan Kuffrey, Attleboro, MA (US); Daniel Allard, Millbury, MA (US)

(21) Appl. No.: 14/939,071

(22) Filed: Nov. 12, 2015

Related U.S. Application Data
(60) Provisional application No. 62/079,749, filed on Nov. 14, 2014.

(51) Int. Cl. A44B 11/06; A61F 5/03; A41F 9/00

(52) U.S. Cl. CPC A44B 11/065; A41F 9/002; A61F 5/03

(57) ABSTRACT

A quick on/off belt with a belt member having two end portions that overlap when the belt is worn around the waist area, a ladder strap fixed to one portion of the belt member, and a ratcheting buckle fixed to another portion of the belt member. The belt is donned by pushing the strap into the buckle and ratcheting the buckle to the desired tightness, and the belt is loosened by operating the buckle so as to release the strap from the buckle.

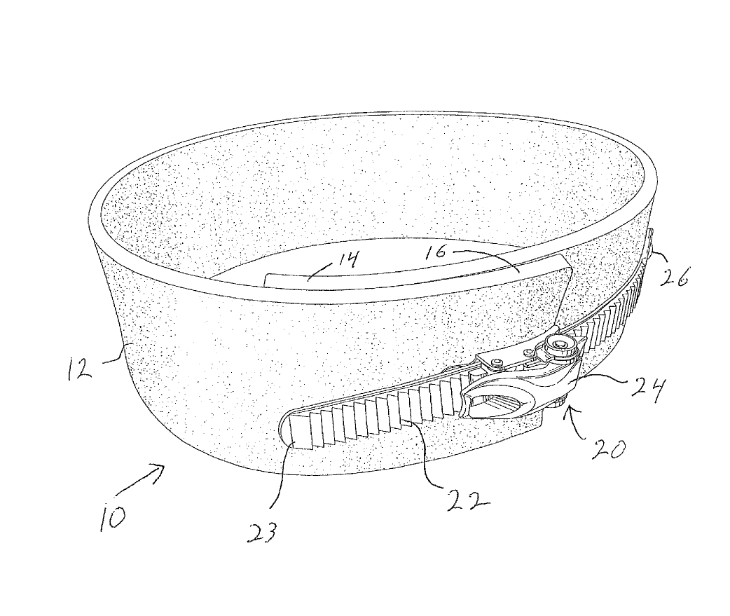

### CROSS-REFERENCE TO RELATED APPLICATION

This application claims priority to Provisional Application Ser. No. 62/079,749, with a filing date of Nov. 14, 2014.

### BACKGROUND

This disclosure relates to a belt.

Weightlifting belts are used in a multitude of sports including but not limited to power lifting and Crossfit. Weightlifting belts are used to force better body mechanics when lifting due decrease in spinal flexion and increase in abdominal cavity pressure, which acts to stabilize the spine. Traditional weightlifting belts are made from a thick leather and are usually about 4 inches wide; this would fall under IPF regulation for size. These belts are made similar to a standard dress belt to hold up pants with the buckle and tongue releasably holding together each end. Other options for weightlifting belts are made of materials such as nylon or neoprene and can come in a variety of widths.

Weightlifting belts should be able to be easily tightened and easily taken off. For it to work efficiently the belt must be able to be tightened and loosened easily by the wearer.

During competitions athletes may have to transition from a weightlifting movement to a movement not requiring a belt. This requires the belt to have the ability to go on and tighten quickly as well as take off easily.

Tool belts also should be easy to put on and take off. They should also remain snug and comfortable under varying weight loads.

### SUMMARY

With the increasing popularity of the sport of fitness it is crucial to be able to utilize a belt as a safety device but also be able to put this belt on and take it off quickly and easily. Also it is desirable to add more support than is afforded by the traditional weightlifting belt by allowing the belt to be tightened in small increments, such as by fraction of inches.

Featured herein in one example is a quick on/off belt (e.g., a weightlifting belt or a tool belt) comprising a belt member having two end portions that overlap when the belt is worn around the waist area, a ladder strap fixed to one portion of the belt member, and a ratcheting buckle fixed to another portion of the belt member. The belt is donned by pushing the strap into the buckle and ratcheting the buckle to the desired belt tightness, and the belt is loosened by operating the buckle so as to release the strap from the buckle. The strap and buckle can be coupled to the belt member at about the midpoint of the width of the belt member. The strap location on the belt member can be adjustable.

### BRIEF DESCRIPTION OF THE DRAWING

The drawing shows a weightlifting belt.

### DETAILED DESCRIPTION

The disclosure herein utilizes a leather, nylon or neoprene belt member, for example. The belt member can have a width of 2″-4″ for use for weightlifting, and typically a width closer to around 2″ for use as a tool belt. Other widths are contemplated, and other uses of the belt are also contemplated. The belt incorporates a ladder strap and ratcheting buckle system for tightening and loosening the belt member. This ladder strap and ratcheting buckle system can replace all traditional tongue and buckle or Velcro straps on weightlifting belts and tool belts.

An example of use as a weightlifting belt is shown in the enclosed figure. Weightlifting belt 10 comprises belt member 12 with end portions 14 and 16; end portions 14 and 16 will overlap when the belt member is worn around the waist area. Ladder strap and ratcheting buckle closure 20 comprise ladder strap 22 which is fixed to belt member portion 14 and ratcheting buckle 24 which is fixed to belt member portion 16. Closure 20 can be a standard closure, for example of the type used in snowboard bindings. Other types of ladder strap and ratcheting buckle closures can be used.

The belt is donned by wrapping belt member 12 around the waist, with portions 14 and 16 overlapping, as shown in the figure. Then, the end 23 of ladder strap 22 is pushed into buckle 24. This is a simple, quick, single-axis motion, as opposed to donning a traditional belt which requires having to thread a free end through a buckle and engage the prong of the buckle frame in a hole in the free end, which is a more complex, multi-axis and time-consuming donning operation. This can cause an athlete to lose valuable time completing a weightlifting maneuver.

The belt can then be tightened by ratcheting buckle 24 until the desired fit is obtained. Because ladder strap 22 allows for small tightening increments of a fraction of an inch, belt 10 can be tightened to different degrees, as desired. Thus the belt is easy to put on and take off, and can be easily adjusted (tightened/loosened) to accommodate different weight loads, comforts and uses. For example, tool belts also should be easy to put on and take off. They should also remain snug and comfortable under varying weight loads.

In order to take the belt off the wearer would press the release mechanism located at the front of the buckle 24. This would allow the ladder strap 22 to be retracted, so as to loosen the belt member a given amount, as warranted by the wearer. The strap can be loosened just enough to take pressure off the wearer but keep it on the body (which might be done during a workout that involves both weightlifting and non-weightlifting movements), or it can be loosened enough to be taken off entirely.

The buckle and ladder strap can be secured to the belt member 12 in a desired fashion, for example using rivets or perhaps a releasable two-part threaded fastener, such as fastener 26 for the strap and another fastener (not shown) for the buckle. The buckle and ladder strap are desirably but not necessarily located in the middle of the width of the belt member; this allows the wearer to wear the belt with either side up and thus be able to operate the buckle with either the right hand or the left hand as desired. Buckle 24 is preferably placed such that it's end is about flush with end 16 of the belt member 12, although the buckle could be located elsewhere.

The length and placement of strap 22 can be chosen to achieve a desired adjustability range of belt 10. In one non-limiting example belt member 12 can be about 41″ long. Strap 22 can be a ladder strap from a snowboard binding, about 11″ long and 1″ wide. Strap 22 can be secured such that about 2″ of its length overhangs from end 14, leaving about 9″ of adjustment range. A 41″ belt member can thus be used on waists of from about 30″ to about 40″. Other lengths of belt members, straps, and strap overhangs can be selected to achieve a desired adjustability range and waist size range. Thus one belt can be used by many people.

Also, the strap could be coupled to the belt in a manner that allowed the strap location to be changed; for example by using an attachment system of threaded belt hole inserts and a machine screw or the like that is received in the insert, along with strap mounting holes located at more than one position on belt end portion 14. This allows the location of fastener 26 to be changed.

Although specific features and aspects are shown and described, these illustrate but do not limit the scope of the invention, which is established solely by the claims.

What is claimed is:

1. A quick on/off belt, comprising:
a belt member having two end portions that overlap when the belt is worn around the waist area;
a ladder strap fixed to one portion of the belt member; and
a ratcheting buckle fixed to another portion of the belt member;
wherein the belt is donned by pushing the strap into the buckle and ratcheting the buckle to the desired belt tightness, and the belt is loosened by operating the buckle so as to release the strap from the buckle.

2. The quick on/off belt of claim 1 wherein the strap and buckle are coupled to the belt member at about the midpoint of the width of the belt.

3. The quick on/off belt of claim 1 wherein the strap location on the belt member is adjustable.

4. The quick on/off belt of claim 1 constructed and arranged to be used as a weightlifting belt.

5. The quick on/off belt of claim 1 constructed and arranged to be used as a tool belt.
